(19) United States
(12) Patent Application Publication
(10) Pub. No.: US 2004/0219210 A1
(43) Pub. Date: Nov. 4, 2004

(54) Controlled release solid dosage nifedipine formulations

(76) Inventor: Jian-Hwa Guo, Hudson, OH (US)

Correspondence Address: NOVEON IP HOLDINGS CORP., 9911 BRECKSVILLE ROAD, CLEVELAND, OH, 44141-3247, US

(21) Appl. No.: 10/829,648

(22) Filed: Apr. 22, 2004

Related U.S. Application Data
(60) Provisional application No. 60/466,923, filed on May 1, 2003.

(51) Int. Cl. A61K 9/22; A61K 9/42

(52) U.S. Cl. 424/468

(57) ABSTRACT

A polymer or copolymer composition derived from one or more unsaturated carboxylic acids that are cross-linked and nifedipine in conjunction with conventional materials such as fillers, excipients, and optional surface active agents is disclosed. Solid dosage forms of sustained release tablets containing the polymer or copolymer composition can be formed by wet granulation followed by mixing with other tableting ingredients, compression to form a tablet core, and finally coating to form the finished tablets. The polymer or copolymer, as a controlled release agent, can enhance controlled-release properties while meeting acceptable release rates as specified by the USP.

### BACKGROUND OF THE INVENTION

1. Field of the Invention

The invention is directed to sustained release formulations of solid dosage nifedipine utilizing a cross-linked polymer or copolymer derived from one or more unsaturated carboxylic acids, which provides sustained release properties at low concentrations, while meeting acceptable release rates as specified by the United States Pharmacopeial Convention (USP).

2. Description of the Prior Art

Nifedipine is a well-known pharmaceutical agent for the inhibition of the passage of calcium ions into vascular smooth muscle and cardiac muscle without altering serum calcium concentrations. Currently, however, there are a limited number of oral therapeutic systems containing nifedipine in solid dosage form.

In U.S. Pat. No. 5,871,775 there is taught a controlled release pharmaceutical composition for the oral administration of nifedipine formed from an amorphous coprecipitate of nifedipine and polyvinylpyrrolidone with suitable excipients. The release rate of the nifedipine may be varied from 8 to 24 hours by varying the amounts of the cellulose derivative, the carboxypolymethylene and the lactose.

In U.S. Pat. No. 5,861,173 there is described a long-lasting release solid nifedipine preparation which exhibits clinically sufficient effect when administered once per day. The invention provides a press coated tablet whose core and shell each contains nifedipine.

The prior art methods of nifedipine delivery, however, have the inherent drawbacks of requiring complex and expensive methods of production and require higher concentrations of nifedipine in order to achieve and maintain a therapeutic range. Additionally, the present invention avoids the use of a combination of povidone and carboxypolymethylene in the same tablet which are known to form a complex under certain circumstances and can affect the drug release profile of the active ingredient.

### SUMMARY OF THE INVENTION

Solid dosage forms of sustained release tablets containing nifedipine are formed by using granules formed by wet granulation mixed with direct compression ingredients; these tablet cores are subsequently coated with an aqueous coating formulation to form the finished tablet. The solid dosage form consists of a polymer or copolymer derived from one or more unsaturated carboxylic acids that is cross-linked and nifedipine in conjunction with conventional materials such as fillers, excipients, and surface active agents. The polymer or copolymer as a sustained release agent can enhance sustained release properties at lower concentrations than prior art systems, while meeting acceptable release rates as specified by the USP.

### BRIEF DESCRIPTION OF THE DRAWING FIGURES

FIG. 1 is a graphic representation of the dissolution profile of a nifedipine tablet of the present invention, along with lines showing the USP requirements.

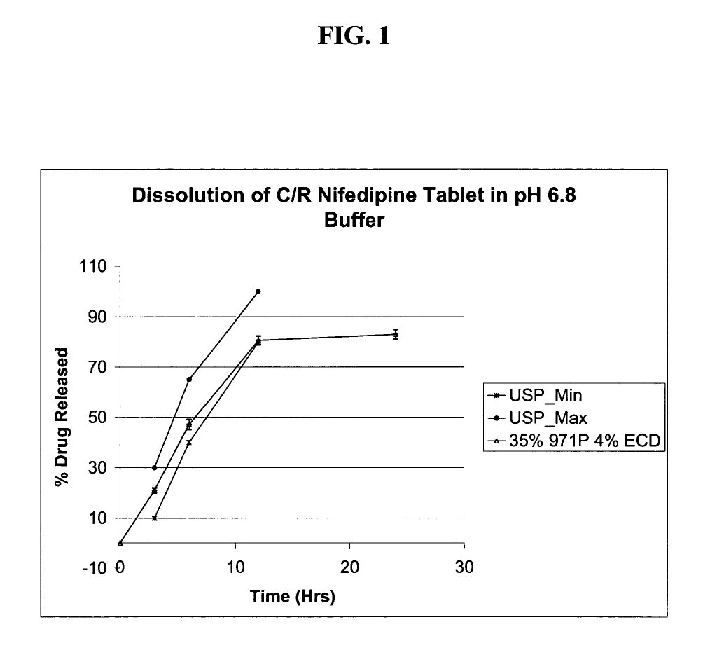

### DESCRIPTION OF THE INVENTION

The polymer or copolymers of the present invention provides sustained release of nifedipine in sustained release formulations, depending upon the choice of ingredients and processing of the formulation. The polymer or copolymers are derived from one or more unsaturated carboxylic acid monomers, (i.e., (di)carboxylic acid) generally having one or two carboxylic acid groups, desirably having one carbon to carbon double bond and containing generally a total of from 3 to about 10 carbon atoms and preferably from 3 to about 5 carbon atoms such as α-β-unsaturated monocarboxylic acids, for example, acrylic acid, methacrylic acid, and crotonic acid, and the like, or dicarboxylic acids such as itaconic acid, fumaric acid, maleic acid, aconitic acid, and the like. Moreover, half ester monomers of such diacids with alkanols containing from 1 to about 4 carbon atoms can also be utilized, such as monomethyl fumarate. Preferred acids include acrylic acid or maleic acid. Additionally, diacids capable of forming cyclic anhydrides, such as maleic, may be polymerized as the anhydride and later reacted with water or alcohols to form the equivalent of maleic acid or monoalkyl maleate copolymer.

Optionally, one or more oxygen-containing unsaturated comonomers having a total of from 3 to about 40 carbon atoms, such as esters of the above unsaturated (di)carboxylic acids, that is, mono or di, especially alkyl esters containing a total of from 1 to about 30 carbon atoms in the alkyl group can also be utilized as comonomers to form the copolymer. Examples of such esters include ethyl acrylate, butyl acrylate, 2-ethylhexyl acrylate, dodecyl acrylate, hexadecyl acrylate, and octadecyl acrylate, and the like, with the C₁₀ to C₃₀ acrylates being preferred.

Another optional class of comonomers are the various anhydrides of the above-noted carboxylic acids such as maleic anhydride, and the like. Moreover, another optional class of suitable comonomers are the various alkyl vinyl ethers wherein the alkyl group contains from 1 to about 20 carbon atoms with examples including ethyl vinyl ether, methyl vinyl ether, and the like. Examples of suitable cross-linked commercially available rheology modifying polymers or copolymers include Carbopol® 941, 934P NF, 971P NF, 981 and 71G NF polymers manufactured by Noveon, Inc., as well as Synthalen® L polymer made by 3V/Sigma, and Aqupec® HV-501 and HV-501E polymers made by Sumitomo Seika. Preferred polymers are 934P NF and 971P NF Carbopol® polymers.

The amount of the one or more oxygen-containing acid comonomers when utilized is generally a minor amount, such as from about 0.01% to about 40% by weight, desirably from about 0.5% to about 35% by weight, and preferably from about 1% to about 25% by weight based upon the total weight of all the rheology modifying polymer or copolymer forming monomers and comonomers. Thus, the amount of the one or more unsaturated (di)carboxylic acid monomers, half ester thereof, or combinations thereof, is generally from about 60% to about 99.99% by weight, desirably from about 65% to about 99.5% by weight, and preferably from about 75% to about 99% by weight based upon the total weight of all rheology modifying polymer or copolymer forming monomers or comonomers.

The various polymers or copolymers of the present invention are generally anhydrous. That is, they generally contain 5 parts by weight or less, desirably 3 parts or 2 parts by weight or less, and preferably 1 part or less by weight, and even nil, that is no parts by weight, of water per 100 parts by weight of the one or more rheology modifying polymers or copolymers.

It is an important aspect of the present invention that the polymer or copolymer be cross-linked with one or more polyunsaturated monomers or comonomers. Suitable cross-linking agents are known to the art and literature and generally include the various allyl ethers of sucrose, pentaerythritol, propylene, or derivatives thereof, or various polyols. Specific examples include diallylphthalate, divinyl glycol, divinyl benzene, allyl(meth)acrylate, ethylene glycol di(meth)acrylate, diallyl itaconate, diallyl fumarate, or diallyl maleate. Derivatives of castor oils or polyols such as esterified with an ethylenically unsaturated carboxylic acid and the like can be used. Preferred cross-linking agents include divinyl glycol, allyl ether of sucrose, allyl ether of pentaerythritol, allyl ether of propylene, diallylphthalate, and combinations thereof.

The amount of the cross-linking agent is from about 0.01 to about 3.5 parts by weight, desirably from about 0.02 to about 2.5 parts by weight, and preferably from about 0.03 to about 1.5 parts by weight per 100 total parts by weight of the one or more rheology monomers or comonomers.

The rheology modifying polymers or copolymers of the present invention are produced by conventional methods known to the art and to the literature such as by dispersion or precipitation polymerization utilizing suitable organic solvents such as various hydrocarbons, esters, halogenated hydrocarbon compounds and the like, with specific examples including aromatic solvents such as benzene, or toluene; various cycloaliphatic solvents such as cyclohexane; various esters such as ethyl acetate and methyl formate, ethyl formate; various chlorinated hydrocarbons such as dichloromethane; and combinations thereof. Preferred solvents generally include benzene, hexane, methylene chloride, blends of ethyl acetate and cyclohexane, or ethyl acetate, and the like.

In addition to containing the rheology modifying polymer or copolymer and nifedipine as active ingredient, the solid dosage formulation will contain various fillers, excipients, surfactants, and the like, as are known to those skilled in the art. The excipients are generally utilized to give a desirable slow release profile as well as other desirable attributes of a solid dosage tablet, including color, hardness, crushing strength, and low friability. Accordingly, such excipients can be one or more of fillers, binders, colorants, coating agents, slow release compounds, and the like.

In order to produce a flowable mixture which contains the cross-linked polymer or copolymer of the present invention, as well as the active ingredient, suitable excipients can include microcrystalline cellulose such as Avicel® PH101, Avicel PH102, Avicel PH200, Avicel PH301, and Avicel PH302 available from FMC Corporation, Vivapur 101, Vivapur 102 available from Rettenmaier and Sohne GMbH, Emcocel 50 M and Emcocel 90 M available from Penwest Company; dicalcium phosphate such as Elcema® available from Degussa; A-Tab®; DiTab® available from Rhodia; lactose monohydrate such as Flow-Lac® 100; Pharmatose® DCL11, Pharmatose DCL15, Pharmatose DCL21 available from DMC International; Tablettose® 80 available from Meggle; and tricalcium phosphate such as Tri-Tab®; Fast Flo Lactose from Foremost; and Prosolve® (Silicified MCC) from Penwest. The amount of one or more excipients utilized will generally be from about 1 to about 90 parts by weight, with from about 5 to about 60 parts by weight of the total dosage formulation being preferred, based upon tablet performance. Higher levels of excipient are generally used with highly active drugs or where only a low dose of drug is being dispensed. This enables the preparation of a tablet which can be easily picked up, handled, counted, etc. Tablets which are too small are difficult to pick up, are easily dropped and lost, and are otherwise inconvenient.

Further excipients utilized are those customarily used in tableting for the preparation of granulates, including binders, lubricants, glidants, dispersants, fillers and the like. Thus, it is possible to include conventional materials such as lactose, saccharose, sorbitol, mannitol, starch, cellulose, or magnesium stearate, in addition to the excipients listed hereinabove.

Optionally, it is contemplated to utilize various solubility enhancers and surface active agents in the practice of the present invention. One class of solubility enhancers which have little surfactant activity is the polyethylene glycol series, such as PEG 600. Other useful types in this series range in molecular weight from 200 to 7,000,000. Suitable surface active agents and solubility enhancers include anionic surfactants such as sodium lauryl sulfate, sodium, potassium or magnesium n-dodecyl sulfate, n-tetradecylsulfate, n-hexadecyl sulfate, n-tetradecyloxyethyl sulfate, n-hexadecyloxyethyl sulfate or n-octadecyloxyethyl sulfate; or sodium, potassium or magnesium n-dodecanesulfonate; sodium, potassium or magnesium n-tetradecanesulfonate, n-hexadecanesulfonate or n-octadecanesulfonate, and the like.

Additional suitable surfactants are non-ionic surfactants of the fatty acid polyhydroxy alcohol ester type such as sorbitan monolaurate, sorbitan monooleate, sorbitan monostearate or sorbitan monopalmitate, sorbitan tri-stearate or trioleate, polyethylene glycol fatty acid ester such as polyoxyethyl stearate, polyethylene glycol 600 stearate, and the like. Further additional surfactants include polyoxyethylene alkyl ethers, polyoxyethylene castor oil derivatives, sorbitan polyoxyethylene fatty acid esters, polyoxyethylene fatty acid esters, and polyoxyethylene stearates, such as defined in “Handbook of Pharmaceutical Excipients,” (American Pharmaceutical Association Pub.; 3^{rd} Ed., 2000).

The polymers or copolymers may be utilized in combination with the active ingredient in either powder or granulated form. When in powder form, the powder mixture containing the active must be granulated to further process properly. Granulation can be accomplished by processes known to the art and in the literature, such as, for example, by roller compaction, by slugging, or utilizing wet methods such as a fluidized bed, or low-shear or high-shear wet granulation. Where the polymer is in granular form, it can either be incorporated with the active before granulation, or combined with the active or granules containing the active just before tableting or capsule filling. The granulated polymer or copolymer desirably has a specific particle size range so that when blended with the nifedipine or granules containing nifedipine, a flowable mixture is produced. This is so the mixture can either be tableted on a high-speed tablet press, or easily filled into capsules on automatic equipment. Desirably, the particle size of the polymer or copolymer powder will be from about 0.1 to about 100 microns, and preferably from about 0.2 to about 40 microns. Desirably, the particle size of the polymer or copolymer granules will be from about 40 to about 1600 microns, and preferably from about 105 to about 840 microns.

The granulated cross-linked rheology modifying polymer or copolymers, nifedipine, as well as the one or more excipients and optional surface active agents can be mixed in any conventional manner to produce a blend. For example, it can be mixed in a shell blender, a Vee blender, a double cone blender, a ribbon mixer, and the like. The polymer or copolymers of the present invention are suitable for producing solid dosage forms by generally all conventional processes, including granulation, grinding, compression, casting in a mold, tableting under pressure, and the like. However, preferred processes for production of the solid dosage form of the present invention are wet granulation and direct compression. In a wet granulation technique, the solid dosage form is prepared in the presence of either a granulation solvent or solution of a granulation binder, as is known in the art. The granulation binder may be the polymer or copolymer of the present invention, or any other polymer known to the art as a granulation binder, such as polyvinyl pyrrolidone, hydroxypropylmethyl cellulose, and the like. Alternatively, the granulation fluid may contain no binders or polymers. In a direct compression method, the mixture containing the polymer or copolymer and active is directly fed into any conventional tablet making machine wherein a desired amount of the mixture or blend is fed through an orifice or opening into a tablet die. The die is closed and compresses the mixture to produce a suitably sized and shaped solid dosage article such as a tablet. This method is more fully described in U.S. patent application Ser. No. 09/559,687, which is incorporated by reference herein.

Polymers suitable for use as coatings in the present invention include, but are not limited to, cellulose acylate, cellulose acetate, cellulose diacylate, cellulose diacetate, cellulose triacylate, cellulose triacetate, mono-, di-, and tri-cellulose alkanylate, mono-, di- and tri-alkenylates, mono-, di- and tri-aroylates, cellulose trivalerate, cellulose trilaurate, cellulose tripalmitate, cellulose trioctanoate, cellulose tripropionate, cellulose diesters, cellulose disuccinate, cellulose dipalmitate, cellulose dioctanoate, cellulose dicarpylate, cellulose actate heptonate, cellulose valerate palmitate, cellulose acetate octonoate, cellulose propionate succinate, cellulose acetate valerate, cellulose acetaldehyde, dimethyl cellulose acetate, cellulose acetate ethylcarbamate, semipermeable polyamylsulfanes, semipermeable urethane, cellulose acetate methylcarbamate, cellulose dimethylaminoacetate, semipermeable sulfonated polystyrenes, semipermeable silicone rubbers, semipermeable styrenes, sulfonated polystyrenes, polyurethanes, polydiethylaminomethylstyrene, cellulose acetate methylcarbamate, ethylcellulose, shellac, polymethylstyrene, polyvinylacetate, semipermeble (polysodium styrenesulfonate), and semipermeable poly(vinylbenzymtrimethyl ammonium chloride).

The invention will be better understood by reference to the following examples which serve to illustrate but not to limit the present invention.

### EXAMPLE OF PREPARATION OF SOLID DOSAGE FORM BY WET

TABLE 1
Solid Dosage Core

| | | | | Actual |
|---|---|---|---|---|
| Code | Ingredients | Source | % w/w | Weight (g) |
| A | Nifedipine USP | Spectrum | 10.0 | 159.1 |
| B | Avicel PH-101 | FMC | 51.3 | 815.6 |
| C | Carbopol 971P | Noveon | 35.0 | 556.5 |
| D | Talc | Aldrich | 0.5 | 8.0 |
| E | *Avicel PH-101 | FMC | 2.7 | 43.0 |
| F | *Mg Stearate | Synpro | 0.5 | 8.0 |

*added after other ingredients were wet granulated, dried, and sized to #20 mesh

Nifedipine USP is weighed and placed in an Erweka Planetary Mixer (Type PRS). While operating the mixer at a speed of 113 rpm, ingredients A, B, C and D were slowly added and mixed for approximately 5 minutes. Deionized water was then added to the formulation in 15 milliliter (ml) increments while mixing at a speed from about 149 rpm to about 205 rpm for a period of 2 minutes between the incremental water additions. The moisture endpoint was determined by observing the appearance of the granulation and by hand-squeezing a handful of the granulation and observing its compaction and cohesion behavior upon this treatment, a technique well known to those skilled in the art. This endpoint occurred in this granulation after the addition of 270 ml. of water.

The wet granulation particles were removed from the mixer and passed through a US Standard #6 mesh. The sized particles were laid out on an aluminum baking tray in a thin layer not to exceed 0.25″ thick and placed in a Blue-M Circulated Air Oven Model OV-55C-2 and dried for at least 8 hours at 60° C. In order to ensure removal of all water from the granules, the granules were removed from the oven, cooled, weighed, and dried further in the oven for an additional hour, followed by cooling and weighing a second time. This process is repeated until there is no weight loss between dryings.

Following drying, the granules are ground through a sieve stack to the desired particle size of 20 mesh. For a 20 mesh particle size, a sieve stack of 8 mesh, 14 mesh, and 20 mesh was utilized.

Ingredient “E” was then added to the dried and sized particles. Mixing was accomplished in four equal batches. To a Patterson-Kelly Twin Shell Mixer was added one fourth (384.8 g) of the dried and sized particles together with 10.75 g of ingredient “E”. The mixture was then mixed for 25 minutes. As each batch was finished, it was placed in a separate container.

Magnesium stearate in an amount of 2.0 g each was then added to each of the above four batches. Mixing was accomplished in a Patterson-Kelly Twin Shell Mixer for two minutes. All the batches were collected into a single large container. After checking the flow index, the mixture was then tableted to form tablet cores.

TABLE 2
Coating Formulation

| Ingredient | Source | Wet Wt. (g.) | Dry Wt. (g.) |
|---|---|---|---|
| Aquacoat ® ECD | FMC | 100.0 | 30.0 |
| Triacetin | Aldrich | 6.05 | 6.05 |
| Deionized Water | | 248.6 | 0 |

The coating formulation was prepared by mixing ingredients from Table 2 in a beaker added in the order as listed, while stirring at 300 rpm. The mixture was stirred for one additional hour using a propeller mixer. This stirring was continued during the coating process, to prevent settling of the dispersed coating polymer.

The tablet cores were coated using a Niro Aeromatic Fielder STREA-1 fluidized bed unit set up in a topspray configuration. The tablets were coated in 2 stages, using the machine settings in Table 3. Three hundred grams of the tablet cores prepared above, along with 100 g of 300-mg. dummy tablet cores, were placed in the fluidized bed unit.

TABLE 3

| | First Coating | First Finishing |
|---|---|---|
| Parameter | Period | Period |
| Time (min.) | 44 | 5 |
| Spray Pressure (bar) | 2.5 | 0 |
| Spray Pump RPM | 2.5-3.25 | 0 |
| Fluidizing Fan Setting | 10-11 | 11 |
| Air Temp. Set Roint (C.) | 42-44 | 50 |
| Measured Inlet Temp. (C.) | 40-46 | 46-47 |
| Measured Bed Temp. (C.) | 29-39 | 46-49 |
| Measured Outlet Temp. (C.) | 27-38 | 42-46 |

The tablets were dried one hour in a circulating-air oven at 50° C. and weighed to determine the add on and required further coating to be added. The tablets were replaced in the fluidized-bed coater and subjected to the following treatment:

TABLE 4

| | Second Coating | Second Finishing |
|---|---|---|
| Parameter | Period | Period |
| Time (min.) | 63 | 2 |
| Spray Pressure (bar) | 2.5 | 0 |
| Spray Pump RPM | 2.5-3.0 | 0 |
| Fluidizing Fan Setting | 10.5-11 | 11 |
| Air Temp. Set Roint (C.) | 42 | 60 |
| Measured Inlet Temp. (C.) | 42-44 | 44-45 |
| Measured Bed Temp. (C.) | 37-40 | 38-40 |
| Measured Outlet Temp. (C.) | 36-39 | 38-39 |

The tablets were further dried and “cured” in an air-circulating oven at 60° C. for 2 hours. The tablets weighed 310.8 grams, for a total coating weight add on of 3.6%.

### Dissolution Testing

USP requirements for dissolution rates of nifedipine are as follows:

| Time (minutes) | Amount Dissolved |
|---|---|
| 180 | Between 10% and 30% |
| 360 | Between 40 and 65% |
| 720 | Not less than 80% |

Dissolution testing was performed using a Hanson Research USP Type 2 (paddle stirrer) dissolution apparatus model SR-8Plus. The tests were conducted at 37° C. in pH 6.8 phosphate buffer, while stirring at 50 rpm. Detection was by HPLC. Results were corrected for the sample volume previously withdrawn.

TABLE 5
(Wet Granulation - Coated Tablets)

Time (minutes)

| | 180 | 360 | 720 | 1440 |
|---|---|---|---|---|
| % Dissolved | 21.05 | 47.11 | 80.59 | 83.00 |
| Std Deviation | 0.91 | 1.87 | 1.66 | 1.82 |

The above results clearly indicate that the solid dosage form of the present invention meets the USP criteria for dissolution rates of sustained release nifedipine.

While in accordance with the Patent Statutes, the best mode and preferred embodiment have been set forth, the scope of the invention is not limited thereto but rather by the scope of the claims.

What is claimed is:

1. A sustained release solid dosage nifedipine composition, comprising:
(a) a rheology modifying polymer composition polymerized from a monomer composition comprising at least one unsaturated (di)carboxylic acid containing monomer having a total of from 3 to about 10 carbon atoms or anhydrides thereof, or at least one half ester monomer of said unsaturated (di)carboxylic acid with an alkanol having from 1 to about 4 carbon atoms, or combinations thereof; a cross-linking agent; and optionally one or more oxygen-containing comonomers having from 3 to about 40 carbon atoms;
(b) nifedipine as an active ingredient;
(c) one or more excipients; and
(d) optionally, a surface active agent and a solubility enhancer and mixtures thereof.

2. A sustained release solid dosage nifedipine composition of claim 1 wherein said carboxylic acid monomer is a monocarboxylic acid monomer selected from acrylic acid, methacrylic acid, crotonic acid, and mixtures thereof.

3. A sustained release solid dosage nifedipine composition of claim 1 wherein said carboxylic acid monomer is a dicarboxylic acid monomer selected from itaconic acid, fumaric acid, maleic acid, aconitic acid, and mixtures thereof.

4. A sustained release solid dosage nifedipine composition of claim 1 wherein said half ester monomer of said unsaturated dicarboxylic acid is monomethyl fumarate.

5. A sustained release solid dosage nifedipine composition of claim 1 wherein said one or more oxygen-containing comonomers is selected from a C₁ to C₃₀ alkyl ester or half ester of a carboxylic acid or a dicarboxylic acid.

6. A sustained release solid dosage nifedipine composition of claim 1 wherein said anhydride of said carboxylic acid monomer is maleic anhydride.

7. A sustained release solid dosage nifedipine composition of claim 5 wherein said one or more oxygen-containing comonomers is selected from ethyl acrylate, butyl acrylate, 2-ethylhexyl acrylate, dodecyl acrylate, hexadecyl acrylate, octadecyl acrylate, and mixtures thereof.

8. A sustained release solid dosage nifedipine composition of claim 1 wherein said monomer composition further comprises a C, to C₂₀ alkyl vinyl ether.

9. A sustained release solid dosage nifedipine composition of claim 8 wherein said alkyl vinyl ether is selected from ethyl vinyl ether and methyl vinyl ether.

10. A sustained release solid dosage nifedipine composition of claim 1 wherein the amount of one or more unsaturated (di)carboxylic acid monomers, anhydrides and half esters thereof, or combinations thereof in said polymerizable monomer composition are present in the amount of from about 60% to about 99.99% by weight, based upon the total weight of all rheology modifying polymer forming monomers.

11. A sustained release solid dosage nifedipine composition of claim 1 wherein said crosslinker is selected from allyl ethers of sucrose, pentaerythritol, propylene, polyol derivatives, and mixtures thereof.

12. A sustained release solid dosage nifedipine composition of claim 1 wherein said crosslinker is selected from diallylphthalate, divinyl glycol, divinyl benzene, allyl(meth)acrylate, ethylene glycol di(meth)acrylate, diallyl itaconate, diallyl fumarate, or diallyl maleate, and mixtures thereof.

13. A sustained release solid dosage nifedipine composition of claim 1 wherein said crosslinker in said polymerizable monomer composition is present in the amount of from about 0.01 to about 3.5 parts by weight based upon the total weight of all rheology modifying polymer forming monomers.

14. A sustained release solid dosage nifedipine composition of claim 1 wherein said excipient is selected from fillers, binders, colorants, coating agents, lubricants, surface active agents and said solubility enhancers, glidants, dispersants, slow release compounds and mixtures thereof.

15. A sustained release solid dosage nifedipine composition of claim 14 wherein said excipient is selected from microcrystalline cellulose, talc, lactose, lactose monohydrate, saccharose, sorbitol, mannitol, starch, cellulose, magnesium stearate, tricalcium phosphate, polyvinyl pyrrolidone, hydroxypropylmethyl cellulose, and mixtures thereof.

16. A sustained release solid dosage nifedipine composition of claim 14 wherein said surface active agent and said solubility enhancer are selected from polyethylene glycol, sodium lauryl sulfate, sodium, potassium or magnesium n-dodecyl sulfate, n-tetradecylsulfate, n-hexadecyl sulfate, n-tetradecyloxyethyl sulfate, n-hexadecyloxyethyl sulfate, n-octadecyloxyethyl sulfate, sodium, potassium or magnesium n-dodecanesulfonate; sodium, potassium or magnesium n-tetradecanesulfonate, n-hexadecanesulfonate, n-octadecanesulfonate, sorbitan monolaurate, sorbitan monooleate, sorbitan monostearate, sorbitan monopalmitate, sorbitan tri-stearate or trioleate, polyethylene glycol fatty acid ester such as polyoxyethyl stearate, polyethylene glycol 600 stearate, polyoxyethylene alkyl ethers, polyoxyethylene castor oil derivatives, sorbitan polyoxyethylene fatty acid esters, polyoxyethylene fatty acid esters, polyoxyethylene stearates, and mixtures thereof.

17. A sustained release solid dosage nifedipine tablet comprising the composition of claim 1.

18. A sustained release solid dosage nifedipine tablet comprising the composition of claim 14.

19. A sustained release solid dosage nifedipine tablet comprising the composition of claim 15.

20. A sustained release solid dosage nifedipine tablet having a coating thereon.

21. A sustained release solid dosage nifedipine tablet of claim 20 wherein said coating comprises a polymer selected from, cellulose acylate, cellulose acetate, cellulose diacylate, cellulose diacetate, cellulose triacylate, cellulose triacetate, mono-, di-, and tri-cellulose alkanylate, mono-, di- and tri-alkenylates, mono-, di- and tri-aroylates, cellulose trivalerate, cellulose trilaurate, cellulose tripalmitate, cellulose trioctanoate, cellulose tripropionate, cellulose diesters, cellulose disuccinate, cellulose dipalmitate, cellulose dioctanoate, cellulose dicarpylate, cellulose actate heptonate, cellulose valerate palmitate, cellulose acetate octonoate, cellulose propionate succinate, cellulose acetate valerate, cellulose acetaldehyde, dimethyl cellulose acetate, cellulose acetate ethylcarbamate, semipermeable polyamylsulfanes, semipermeable urethane, cellulose acetate methylcarbamate, cellulose dimethylaminoacetate, semipermeable sulfonated polystyrenes, semipermeable silicone rubbers, semipermeable styrenes, sulfonated polystyrenes, polyurethanes, polydiethylaminomethylstyrene, cellulose acetate methylcarbamate, ethylcellulose, shellac, polymethylstyrene, polyvinylacetate, semipermeble (polysodium styrenesulfonate), and semipermeable poly(vinylbenzymtrimethyl ammonium chloride).

22. A sustained release solid dosage nifedipine tablet comprising the composition of claim 16.

23. A sustained release solid dosage nifedipine composition comprising the composition of claim 1 in the form of granules.

24. A sustained release solid dosage nifedipine composition comprising the composition of claim 14 in the form of granules.

25. A sustained release solid dosage nifedipine composition comprising the composition of claim 15 in the form of granules.

26. A sustained release solid dosage nifedipine composition comprising the composition of claim 16 in the form of granules.
